(19) United States
(12) Patent Application Publication
(10) Pub. No.: US 2012/0010667 A1
(43) Pub. Date: Jan. 12, 2012

(54) FRACTURE-SPECIFIC DISTAL RADIUS PLATES

(76) Inventor: W. Andrew Eglseder, Owings Mills, MD (US)

(21) Appl. No.: 13/257,008

(22) PCT Filed: Mar. 23, 2010
(86) PCT No.: PCT/US10/28209

§ 371 Date: Sep. 16, 2011

Related U.S. Application Data
(60) Provisional application No. 61/162,471, filed on Mar. 23, 2009.

(51) Int. Cl. A61B 17/80; A61B 17/88

(52) U.S. Cl. 606/286

(57) ABSTRACT

The invention provides an improved distal radius locking plate having a body comprising a shaft portion and a head portion. The shaft portion is adapted for receiving bone screws to fix the bone plate to a shaft of the radius. The head portion includes a first head section and a second head section. The first and second head sections extend laterally away from a longitudinal axis of the shaft portion in generally opposite directions, generally forming a T-shape. The first and second head sections are configured and arranged to secure a plurality of bone screws in the radius. Further, the plate may be configured with one or more prongs that can attach to small bone fragments. The plate of the present invention can be used as a guide in reducing the fracture prior to fixation since the plate has a precontoured shape matching the shape of an unfractured distal radius. The plate of the present invention may be sized and configured to match the contour of the palmar lip of the lunate facet. Alternatively, the plate of the present invention may be sized and configured to match the contour of the floor of the fourth extensor compartment.

### TECHNICAL FIELD

The present invention relates generally the treatment of fractures and to devices used for stabilizing bone fractures, and more particularly the invention relates to bone plates and even more particularly the invention relates to a bone plate for fixating specific fractures of a distal radius bone.

### BACKGROUND ART

The radius is one of two long bones found in the human forearm. The radius, like other bones, is susceptible to a variety of fractures and other dislocations. For example, fractures of the radius are a common result of forward falls, with the palms facing downward. In such falls, force exerted on the hands and wrist at impact frequently produces displacement of one or more bone fragments created distal to the fracture site.

Alignment and fixation of a metaphyseal fracture (occurring at the extremity of a shaft of a long bone) are typically performed by one of several methods: casting, external fixation, pinning, and plating. Casting is non-invasive, but may not be able to maintain alignment of the fracture where many bone fragments exist. Therefore, as an alternative, external fixators may be used. External fixators utilize a method known as ligamentotaxis, which provides distraction forces across the joint and permits the fracture to be aligned based upon the tension placed on the surrounding ligaments. However, while external fixators can maintain the position of the wrist bones, it may nevertheless be difficult in certain fractures to first provide the bones in proper alignment. In addition, external fixators are often not suitable for fractures resulting in multiple bone fragments. Pinning with K-wires (Kirschner wires) is an invasive procedure whereby pins are positioned into the various fragments. This is a difficult and time-consuming procedure that provides limited fixation if the bone is comminuted or osteoporotic. Plating utilizes a stabilizing metal plate typically placed against the dorsal side of a bone, and screws extending from the plate into holes drilled in the bone fragments to provide stabilized fixation of the fragments. However, many currently available plate systems fail to provide desirable alignment and stabilization.

Fractures of the radius may be treated by exposing the fracture site and reducing the bone fracture and then placing a plate or other means onto the bone to fixate the fracture for healing in the reduced position. Reducing the fracture includes realigning and positioning the fractured portions of the bone to their original position or similar stable position. Fixating the fracture includes positioning a plate over the fractured portions and securing the plate onto the fractured bones and adjacent nonfractured bones with bone screws.

Where a single fracture occurs, the original position of the fractured portion can be easily recognized and the unfractured portion functions as a guide or reference point for reducing and fixating the fractured portion to the unfractured bone portion. However, this challenge is more acute with multiple part fractures, and fracture dislocations since no single portion of the bone remains unfractured. Therefore, no single portion of the fractured bone can act as a stable guide or reference to insure the return of the fractured portions to their proper position and to remain stable to enable proper reduction and fixation of the multiple fractured portions at the same time.

Examples of a bone fixation plates are described in U.S. Pat. Nos. 6,712,820 and 7,294,130, both to Orbay and U.S. Pat. No. 6,221,073 to Weiss et al., all of which disclose a bone plate apparatus for use with fixation of wrist fractures. The plate is positioned on the dorsal or volar side of the radial bone and a plurality of bone screws secure the plate along a non-fractured portion of the bone.

Another example of a bone fixation device of the prior art is described in U.S. Pat. No. 6,283,969 to Grusin et al., which discloses a distal radial plate having a relatively wide T-shape. The longitudinal sections of the plate contain a plurality of recessed holes for insertion of a bone screw. The plate is designed to be bent along its longitudinal or transverse segments of the plate.

U.S. Pat. No. 6,096,040 to Esser discloses a bone plate especially adapted for repairs of the proximal humerus bone. The plate is configured and arranged to match the contour of an unfractured proximal humerus. A plurality of holes and slots is provided to secure multiple fractures of the head.

U.S. Pat. No. 7,354,441 to Frigg and U.S. Patent Application Publication No. 2007/0233114 by Bouman describe bone plates having particularly configured holes and slots for attaching and locking bones screws to the plate and to the bone under repair. In some cases, the slot may be configured to engage threads on the head of a bone screw; alternatively, the slot may be configured to cooperate with the head of a different bone screw.

While known bone plates have been configured for holding bone fragments in place, none of the prior art discloses a bone plate especially configured with one or more prongs to secure bone fragments. It would be desirable to provide a bone fixation device for use with the specific fractures of the distal end of the radius. Moreover, a fixation device should provide desirable alignment and stabilization of the bone structure of the distal radius.

### DISCLOSURE OF INVENTION

Accordingly, it is an object of the present invention to provide a distal radius locking plate that avoids the disadvantages of the prior art.

It is an object of the present invention to provide a distal radius locking plate that enables fixation of fractures of the distal radius. A related object of the present invention is to provide a distal radius locking plate that is specifically contoured to the palmar lip of the lunate facet. Another related object of the present invention is to provide a distal radius locking plate that is specifically contoured to fit the floor of the fourth extensor compartment. A further related object of the present invention is to provide different palmar and dorsal plates to allow implant placement to match the individual fracture pattern.

Another object of the present invention is to provide a distal radius locking plate that is held in place by locking screws. A further object of the present invention is to provide a distal radius locking plate optionally having one or more prongs that can be used to secure bone fragments.

These and other objects of the present invention are accomplished by providing an improved distal radius locking plate having a body comprising a shaft portion and a head portion. The shaft portion is adapted for receiving bone screws to fix the bone plate to a shaft of the radius. The head portion includes a first head section and a second head section. The first and second head sections extend laterally away from a longitudinal axis of the shaft portion in generally opposite directions, generally forming a T-shape. The first and second head sections are configured and arranged to secure a plurality of bone screws in the radius. Further, the plate may be configured with one or more prongs that can attach to small bone fragments. The plate of the present invention can be used as a guide in reducing the fracture prior to fixation since the plate has a precontoured shape matching the shape of an unfractured distal radius. The plate of the present invention may be sized and configured to match the contour of the palmar lip of the lunate facet. Alternatively, the plate of the present invention may be sized and configured to match the contour of the floor of the fourth extensor compartment.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a plan view of a locking plate for the palmar side of a distal radius according to an embodiment of the present invention.

FIG. 2 is a plan view of a locking plate for the dorsal side of a distal radius according to an embodiment of the present invention.

FIG. 3 is a side view of a distal radius showing the contours of the locking plates according to an embodiment of the present invention.

### BEST MODE(S) FOR CARRYING OUT THE INVENTION

The invention provides a bone plate especially adapted for fixing fractures of the distal radius. The bone plate is pre-shaped to match the contour of the anatomic profile of an unfractured distal radius to assist in reduction of complicated fractures.

The plate of the present invention is generally T-shaped; defining an elongate body section and a generally transverse head, and includes a first side that is intended to contact the bone, and a second side opposite the first side. The body section includes a plurality of screw holes for the extension of bone screws therethrough, and optionally one or more substantially smaller K-wire alignment holes. The body section may have a contoured profile that provides a stable shape to prevent rocking of the plate on the bone and maintains anatomical alignment between the fracture fragments. Furthermore, the plate may include one or more prongs that can attach to small bone fragments. Different palmar and dorsal plates allow implant placement to match specific fracture patterns and bone shapes.

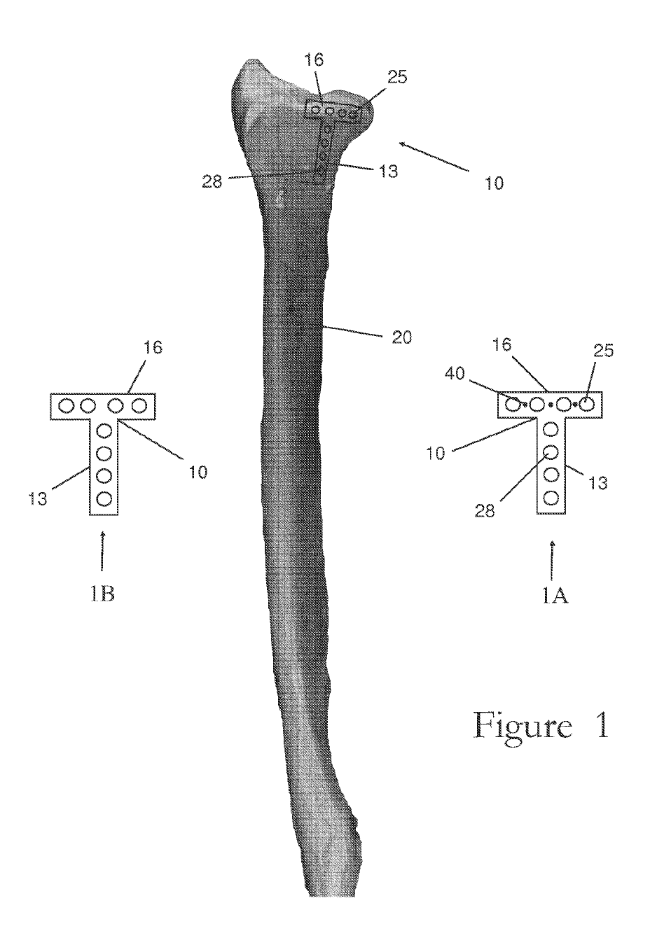

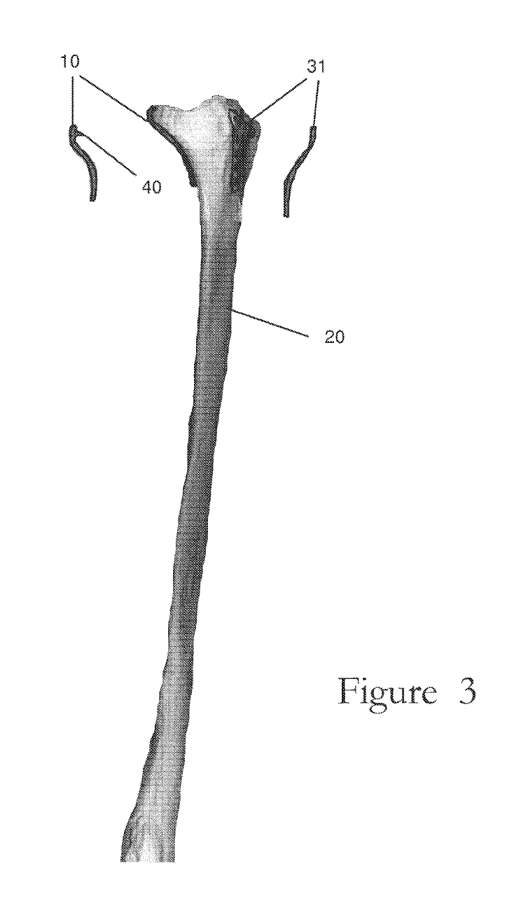

Referring to the drawings, FIG. 1 shows the palmar side of a distal radius having a locking plate, indicated generally as 10, according to the present invention. The plate 10 includes an elongate body 13 and a transverse head 16. The elongate body 13 is connected on one end to the transverse head 16. The elongate body 13 is adapted for receiving a plurality of bone screws to fix the locking plate 10 to a portion of the radius 20. Additionally, the transverse head 16 is adapted for receiving a plurality of bone screws configured and arranged to secure the bone screws implanted in the radius 20. The portion of the figure labeled 1A shows the first side of the plate 10 that is intended to contact the radius 20. The portion of the figure labeled 1B shows the second side of the plate 10, which is opposite the first side. As shown in FIG. 3, the plate 10 should be contoured to match the shape of the palmar lip of the lunate facet.

The plate 10 has a plurality of openings 25. Openings for the extension of bone screws therethrough may be provided in the transverse head 16 and in the elongate body 13. Such bone screws may comprise 1.3 mm or 1.5 mm screws. Other sizes may be used. The screws may be locking screws or other appropriate types of screws. In some embodiments, the openings 28 in the elongate body 13 may comprise slots and are adapted for receiving appropriate fasteners in the radius 20.

The plate 10 can include one or more prongs, such as prong 40, best seen in FIG. 3. The one or more prongs can be variously situated on the transverse head 16 of the plate 10. For example, the prongs can be (but need not be) located between openings 25 for receiving screws on the transverse head 16, at a surface of the transverse head 16 intended to contact the radius 20. In one exemplary embodiment, the transverse head 16 can contain four openings 25 for receiving screws and three prongs 40, whereby each prong is situated between two openings for receiving screws. Other arrangements of prongs can also be used. The transverse head can contain, e.g., one, two, three, four, or more prongs 40 as needed, arranged in any useful configuration on the transverse head 16 so as to contact and secure small bone fragments during use of the plate 10 for stabilization of fractures (such as intra-articular fractures) of the distal radius.

In one example, the one or more prongs 40 can be, e.g., about 2 to about 3 millimeters in length. The one or more prongs 40 can be any shape that would allow the one or more prongs to be at least partially embedded into a bone fragment. Examples of shapes for the one or more prongs 40 are not limited to, approximately cylindrical, approximately conical, or approximately pyramidal.

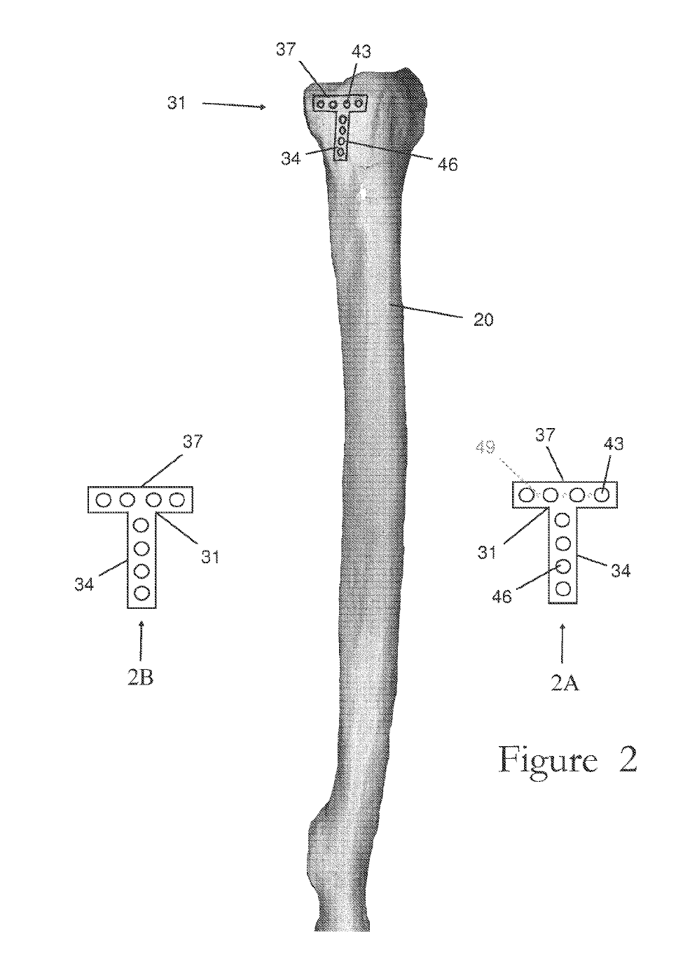

FIG. 2 shows the dorsal side of a distal radius having a locking plate, indicated generally as 31, according to the present invention. The plate 31 includes an elongate body 34 and a transverse head 37. The elongate body 34 is connected on one end to the transverse head 37. The elongate body 34 is adapted for receiving a plurality of bone screws to fix the locking plate 31 to a portion of the radius 20. Additionally, the transverse head 37 is adapted for receiving a plurality of bone screws configured and arranged to secure the bone screws implanted in the radius 20. The portion of the figure labeled 2A shows the first side of the plate 31 that is intended to contact the radius 20. The portion of the figure labeled 2B shows the second side of the plate 31, which is opposite the first side. As shown in FIG. 3, the plate 31 should be contoured to fit the floor of the fourth extensor compartment. The plate 31 may include one or more prongs, such as prong 49, to secure small bone fragments as described for plate 10 above.

The plate 31 has a plurality of openings 43. Openings for the extension of bone screws therethrough may be provided in the transverse head 37 and in the elongate body 34. Such bone screws may comprise 1.5 mm or 2.0 mm screws. Other sizes may be used. The screws may be locking screws or other appropriate types of screws. In some embodiments, the openings 46 in the elongate body 34 may comprise slots and are adapted for receiving appropriate fasteners in the radius 20.

By way of non-limiting example, the elongate body 13, 34 can be about 1.5 centimeters to about 3 centimeters in length, although other sizes can be used as appropriate for the dimensions of any particular patient's distal radius. Similarly, by way of non-limiting example, the transverse head 16, 37 can be about 1.5 centimeters in length, although other sizes can be used as appropriate for the dimensions of any particular patient's distal radius. In one embodiment, locking plates 10 and 31 present a low profile to minimize potential for tendon and soft tissue irritation.

In use, the radius locking plates 10 and 31 can be used separately or in combination for stabilization of fractures of the distal radius, e.g., intra-articular fractures of the distal radius. The elongate body 13, 34 and the transverse head 16, 37 are positioned on the radius 20 across the fracture. The one or more prongs 40 are embedded in small bone fragments on the palmar side of the fracture to hold them in place. If included, one or more prongs 49 are embedded in small bone fragments on the dorsal side of the fracture to hold them in place. A drill, or other appropriate device, is used to make holes in the radius 20. In addition to the prong 40, 49 (if included), appropriate fixing devices, such as surgical screws, sized and configured for use as described, are inserted transversely of the elongate body 13 and transverse head 16, and pass through at least part of the radius 20 to be treated; the line of insertion of the fixing devices being defined by the openings 25, 28, 43, and 46 in the transverse head 16, 37 and the elongate body 13, 34. Optionally, one or more K-wires may also be used.

### INDUSTRIAL APPLICABILITY

The present invention is applicable to providing a device for stabilizing fractures of the distal radius. The invention discloses an anatomically shaped locking plate having one or more prongs of the face of the locking plate that contacts the bone. The one or more prongs can grasp and hold small bone fragments.

1. A distal radius locking plate, comprising:
an elongate body having a plurality of openings adapted for receiving one or more bone screws therethrough; and
a transverse head on an end of said elongate body, said transverse head having a plurality of openings adapted for receiving one or more bone screws therethrough;
wherein said plate is sized and configured to match the palmar lip of the lunate facet or to fit the floor of the fourth extensor compartment; and
wherein said plate sized and configured to match the palmar lip of the lunate facet further comprises one or more prongs, said one or more prongs being sized and configured to secure small bone fragments.

2. (canceled)

3. The locking plate according to claim 1, wherein said plate sized and configured to fit the floor of the fourth extensor compartment further comprises one or more prongs, said one or more prongs being sized and configured to secure small bone fragments.

4. (canceled)

5. The locking plate according to claim 1, said transverse head comprising:
a first portion extending laterally outward from and generally perpendicular to a longitudinal axis of the elongate body; and
a second portion that extends laterally outward from a longitudinal axis of the elongate body portion in a generally opposite direction than the first portion.

6. The locking plate according to claim 5, wherein said plate is generally T-shaped.

7. The locking plate according to claim 1, wherein said elongate body is configured and arranged to secure a plurality of bone screws implanted in the radius.

8. The locking plate according to claim 1, wherein said transverse head is configured and arranged to secure a plurality of bone screws implanted in the radius.

9. A method of stabilizing a distal radial fracture, comprising:
providing a locking plate comprising:
an elongate body having a plurality of openings adapted for receiving one or more bone screws therethrough; and
a transverse head on an end of said elongate body, said transverse head having a plurality of openings adapted for receiving one or more bone screws therethrough;
wherein said plate is sized and configured to match the palmar lip of the lunate facet or to fit the floor of the fourth extensor compartment; and
wherein said plate sized and configured to match the palmar lip of the lunate facet further comprises one or more prongs, said one or more prongs being sized and configured to secure small bone fragments;
aligning said plate across the distal radial fracture;
forming a plurality of holes in the radius; and
anchoring the locking plate to the radius using a plurality of bone screws.

10. (canceled)

11. The method according to claim 9, further comprising the steps of securing any small bone fragments on a palmar side of said fracture with said one or more prongs on the plate sized and configured to match the palmar lip of the lunate facet before anchoring the locking plate to the radius.

12. The method according to claim 9, wherein said plate sized and configured to fit the floor of the fourth extensor compartment further comprises one or more prongs, said one or more prongs being sized and configured to secure small bone fragments.

13. (canceled)

14. The method according to claim 12, further comprising the steps of securing any small bone fragments on a dorsal side of said fracture with said one or more prongs on the plate sized and configured to fit the floor of the fourth extensor compartment before anchoring the locking plate to the radius.

15. The method according to claim 9, said locking plate being pre-shaped to match the contour of the anatomic shape of an unfractured distal radius.

16. The method according to claim 9, said transverse head comprising:
a first portion extending laterally outward from and generally perpendicular to a longitudinal axis of the elongate body; and
a second portion that extends laterally outward from a longitudinal axis of the elongate body portion in a generally opposite direction than the first portion.

17. The method according to claim 9, wherein said elongate body is configured and arranged to secure a plurality of bone screws implanted in the radius.

18. The method according to claim 9, wherein said transverse head is configured and arranged to secure a plurality of bone screws implanted in the radius.

19. A method of stabilizing a distal radial fracture, comprising:
providing a first locking plate comprising:
an elongate body having a plurality of openings adapted for receiving one or more bone screws therethrough;
a transverse head on an end of said elongate body, said transverse head having a plurality of openings adapted for receiving one or more bone screws therethrough; and
one or more prongs, said one or more prongs being sized and configured to secure small bone fragments,
wherein said plate is sized and configured to match the palmar lip of the lunate facet;
providing a second locking plate comprising:
an elongate body having a plurality of openings adapted for receiving one or more bone screws therethrough, and
a transverse head on an end of said elongate body, said transverse head having a plurality of openings adapted for receiving one or more bone screws therethrough;
wherein said plate is sized and configured to fit the floor of the fourth extensor compartment;
aligning said first and second locking plates across the distal radial fracture;
forming a plurality of holes in the radius; and
anchoring the first and second locking plates to the radius using a plurality of bone screws.

20. (canceled)

21. The method according to claim 19, further comprising the steps of securing any small bone fragments on a palmar side of said fracture with said one or more prongs on the plate sized and configured to match the palmar lip of the lunate facet before anchoring the first locking plate to the radius.

22. The method according to claim 19, wherein said plate sized and configured to fit the floor of the fourth extensor compartment further comprises one or more prongs, said one or more prongs being sized and configured to secure small bone fragments.

23. (canceled)

24. The method according to claim 22, further comprising the steps of securing any small bone fragments on a dorsal side of said fracture with said one or more prongs on the plate sized and configured to fit the floor of the fourth extensor compartment before anchoring the second locking plate to the radius.

25. The method according to claim 19, said first and second locking plates being pre-shaped to match the contour of the anatomic shape of an unfractured distal radius.

26. The method according to claim 19, said transverse head of each of said first and second locking plates comprising:
a first portion extending laterally outward from and generally perpendicular to a longitudinal axis of the elongate body; and
a second portion that extends laterally outward from a longitudinal axis of the elongate body portion in a generally opposite direction than the first portion.

27. The method according to claim 19, wherein said elongate body of each of said first and second locking plates is configured and arranged to secure a plurality of bone screws implanted in the radius.

28. The method according to claim 19, wherein said transverse head of each of said first and second locking plates is configured and arranged to secure a plurality of bone screws implanted in the radius.
